(19) United States
(12) Patent Application Publication
(10) Pub. No.: US 2008/0248172 A1
(43) Pub. Date: Oct. 9, 2008

(54) NUTRITIONAL SUPPORT FOR SERVINGS

(75) Inventors: Douglas Ashley BALENTINE, Harriman, NY (US); Tracy RAMIREZ, Edgewater, NJ (US); Sheila Marie WINSHIP, Monroe, NY (US); Kenneth GIMELLI, Waldwick, NJ (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 11/696,208

(22) Filed: Apr. 4, 2007

(51) Int. Cl. G01N 33/02

(52) U.S. Cl. 426/231

(57) ABSTRACT

A method for substantiating servings based on nutritional guidelines and formulating food products to nutritional guidelines is disclosed. Preferably, a method for substantiating fruit and/or vegetable servings and formulating based on nutritional guidelines for fruit and/or vegetables is disclosed.

### FIELD OF THE INVENTION

The present invention is directed to substantiating servings based on nutritional guidelines and formulating food products to nutritional guidelines. More specifically, the present invention is directed to substantiating fruit and/or vegetable servings and formulating based on nutritional guidelines for fruit and/or vegetables.

### BACKGROUND OF THE INVENTION

U.S. Dietary Guidelines and the Canada's Food Guide to Healthy Eating focus on increasing fruit and vegetable consumption to improve health and wellness. Fruits and vegetables are important for meeting the daily value for many key nutrients. Obtaining daily needs for many nutrients is not possible while keeping calories within a range for a healthy weight without consuming fruits and vegetables. Fruit and vegetable intake is also a key factor for providing both bulk and fiber to the diet while not being calorie dense. In both the U.S. and Canada the recommendation is that people consume 4 (½ cup) portions of fruit and 5 (½ cup) portions of vegetables (selecting from all the color groups) per day. In the U.S. legumes are a part of the vegetable group while in Canada they are not. This level of fruit and vegetable consumption is recommended to provide specific nutrient needs as part of a balances healthy diet outline in MyPyramid (http://www.mypyramid.gov/index.html) and by Canada's Food Guide to Healthy Eating.

More particularly, any vegetable/fruit or 100% vegetable/fruit juice counts as a member of the vegetable/fruit group as defined in the US My Pyramid and by Canada's Food Guide to Healthy Eating. Vegetable and fruits may be raw or cooked; fresh, frozen, canned, or dried/dehydrated; and may be whole, cut-up or mashed. In the U.S. and Canada, a serving of vegetables is set at ½ cup, with some exceptions. For Green Leafy vegetables and lettuce the serving size is 1 cup. A serving of fruit is set as ½ cup or an appropriate portion of size of whole or cut fruit. Vegetable/fruit juice or vegetable/fruit purees (½ cup) counts as one serving of vegetables/fruit. A serving size of dehydrated vegetables/fruit is set at ¼ cup. Servings are first set up by volume, defined as a ½ cup portion of most fruits or vegetables. A ½ cup portion of fruit or vegetable juice/puree can replace a serving of solid fruit or vegetables.

Since all fruits and vegetables contain a significant amount of water it would be possible to have many forms of fruits and vegetables, juices and purees that have significantly less water and more solids than typical forms. It would be expected that a portion of fruit or vegetables from these forms would be significantly less than a ½ cup size.

As discussed so far, the number of servings of fruits and vegetables can be defined using the conventional principles outlined above either alone or in combination. One approach is Volume Based on Food Standards (½ cup fruits, vegetables, juice, puree or about ¼ cup dried/dehydrated fruit or vegetable). In this case, concentrated juices or purees would count as a serving if, when unfolded by addition of the water removed in concentration, the volume is restored to a ½ cup portion. Another approach is Solids Content expected from the amount of Fruits and Vegetables making up a serving (although measurements on solids basis use refractive index measurements, so that sugar tends to deliver the solids count but not necessarily a measure of nutrients present in the composition).

As part of delivering a serving of vegetables it is important that the serving not only deliver solids but also the key nutrients expected from the fruit and/or vegetables in the product. Also, in the case of a preparation formulated using concentrates of fruits and vegetable purees or juices, rather than normal strength forms of these ingredients, it can be argued that defining servings of fruits and vegetables contained in the preparation based on conventional principles is misleading.

Therefore, to arrive at the method of the present invention, nutrient delivery, or nutrient density, was the criteria developed to set the recommended daily intake goals for fruits and vegetables based on the Report of the Dietary Guidelines Advisory Committee on Dietary Guidelines for Americans, 2005.

### SUMMARY OF THE INVENTION

The present invention is based on the principle of defining servings of healthy foods based on nutrients, rather than based on volume. Nutrient density based on delivering key nutrients expected from one or more portions of fruits and vegetables based on food composition tables, such as those of the USDA National Nutrient Database found at http://www.nal.USDA.gov/fnic/foodcomp/search/index.html, and from analysis of the actual product is the approach to substantiating food servings according to the present invention. The term nutrient density is used herein to mean a measure of nutrient content per unit volume as compared with per recommended serving size.

According to the rationale on which the present invention is based, starting with the USDA guidelines, for example, the nutritional profile delivered by the fruit and vegetable recommendations is determined. The following nutrients are to be delivered from the fruit and vegetables in an amount of more than about 45% of the daily recommended amount: vitamins A, B6 and C; thiamin; folate; magnesium; copper; potassium; and dietary fiber. Of this list, fruit and vegetables are major sources of vitamins A and C; folate, potassium, and dietary fiber.

The term “comprising” is used herein in it ordinary meaning and means including, made up of, composed of, consisting and/or consisting essentially of. In other words, the term is defined as not being exhaustive of the steps, components, ingredients, or features to which it refers.

The terms fruit and/or vegetables are used herein in their ordinary meaning and mean real fruit and/or vegetables, or juices, purees, concentrates derived from fruit and/or vegetables, regardless of form.

The term ½ cup refers to a fluid measure equivalent to 4 fluid ounces.

The term nutrient density is used herein to mean a measure of nutrient content per unit volume as compared with per recommended serving size.

The term “servings” is used herein to mean food servings and/or portions as recommended by a food regulatory body. By way example but not limitation, servings and/or portions may refer to the USDA recommended daily intake of fruits and/or vegetables, i.e. up to 4 servings of fruits and 5 servings of vegetables.

The term “equivalent” used herein in connection with servings means equivalent to food regulatory body recommended servings as determined by the nutrition density methodology in accordance with the present invention.

Except in the operating and comparative examples, or where otherwise explicitly indicated, all numbers in this description indicating amounts or ratios of material or conditions of reaction, physical properties of materials and/or use are to be understood as modified by the word “about”.

The inventive method for determining number of fruit and vegetable servings in a concentrated fruit and vegetable food preparation includes:

a. providing a volume of said preparation that is up to about 1 cup;

b. measuring nutrient density of said volume;

c. comparing said nutritional density to that in food regulatory body, such as the USDA in the U.S., recommended fruit and vegetable servings;

d. claiming on the package the appropriate equivalent fruit and/or vegetable servings.

Preferably, the number of servings of fruit and/or vegetable to be claimed on food or drink products as packaged is about 3 to about 5, and may even deliver all the recommended daily servings of fruits and vegetables. In a preferred embodiment, the fruit and vegetable preparation is drinkable.
In another aspect, the present invention is a method of formulating a packaged food product comprising nutrient containing food ingredients to provide servings of a particular food group based on nutritional requirements of regulatory bodies, said method comprising:
   a. ascertaining nutritional regulations for at least one nutrient;
   b. measuring the content of said at least one nutrient in said food product;
   c. adjusting the amount of one or more of said food ingredients so as to meet said nutritional regulations for said at least one nutrient;
   d. claiming on the package the equivalent said food group servings.

Preferably, the food product is a fruit and vegetable preparation, a dehydrated side dish, or chilled side dish or meal or any product form. The food group is selected from the group consisting of fruit, vegetable, fat, oil, grain, carbohydrate, and meat, as well as any food group as designated by a regulatory food body, such as the Food Pyramid. Preferably, the number of servings of fruit and/or vegetable to be claimed on food or drink products as packaged is about 3 to about 5. The claiming is preferably based on an equivalent to a freshly picked food. The claiming can also be based on an equivalent to the maximum values for nutrients in USDA National Nutrient Database found at http://www.nal.USDA.gov/fnic/foodcomp/search/index.html or those of other food regulatory bodies.

In a further aspect, the present invention is directed to a method of formulating a food composition to a nutritional target by selecting food ingredients having a plurality of nutrients therein, comprising:
(a) Setting a nutritional target comprising number of servings of a food regulatory body recommended food;
(b) Determining the amount of each nutrient expected from said nutritional target from a food from food composition tables of a food regulatory body;
(c) Adjusting the nutrient list to include only those nutrients above 2% of the daily value based on said food composition tables, thereby determining a key nutrient list; and
(d) Determining an amount of each key nutrient needed to meet said nutritional target;
(e) Measuring the amount of said key nutrients in said food composition by standardized analytical testing;
(f) Adjusting the food composition amount of food ingredients to meet said nutrition target based on said analytical testing.

The target may be for one or more servings of said recommended food. The recommended food may be fruit and/or vegetables, such that the food product may be formulated to provide one or more fruit and/or vegetable servings

In a still further aspect, the present invention is directed to a method for determining the number of fruit and/or vegetable servings in a packaged dehydrated or frozen food composition containing fruit and/or vegetable pieces, said method comprising:

a. providing said composition having a volume of said fruit and/or vegetable pieces that is up to about ¼ cup;

b. measuring the nutrient density of said volume;

c. comparing said nutrient density to that in a food regulatory body recommended fruit and vegetable servings;

d. claiming on the package the appropriate equivalent of fruit and/or vegetable servings.

The number of fruit and/or vegetable servings may be about 1 to about 5, and up to the total daily recommended number of servings The food regulatory body may be USDA and/or Canada Food Guide and/or World Health Organization (WHO) and/or an equivalent regulatory body in a respective territory. It is generally accepted that a healthy diet should be high in fruits and vegetables.

### DETAILED DESCRIPTION OF THE INVENTION

While consumers recognize and acknowledge the need for a high intake of fruits and vegetables, as well as recommendations as to other food groups for a healthy diet and lifestyle, they wish for convenience. Therefore, the present invention is based on the need to provide a number of nutrients toward food regulatory recommended amounts in a packaged food product. Regardless of product or ingredient form, the goal is to provide a nutrient density equivalent number of servings of a food toward a recommended intake amount. For example, if 4 servings of fruits and 5 servings of vegetables is the recommended daily intake, the goal is to provide a food product that provides at least 3 fruit and/or vegetable servings regardless of product form, size, weight or volume.

The method of the present invention was developed with a view to a product in the form of a fruit and vegetable preparation packaged in a bottle that provides up to about ½ cup of fruit and vegetable preparation prepared from a combination of juice concentrates and purees, as well as optionally liquefied, extract or juice forms. Preferably, at least two species of fruits and/or vegetables are used. Since the product is concentrated and drinkable, it is considered a fruit and vegetable preparation rather than a beverage. While so developed, the method of the present invention is not limited to a particular product or product form. While the method is particularly applicable to providing multiple servings of fruits and vegetables in a volume of preparation that is up to about ½ cup, a soup may be prepared using multiple such volumes, e.g., up to 1 cup. Additionally, as an example, the method is applicable, among other products, to dehydrated side dishes containing a dehydrated vegetable component and a carbohydrate component such as rice or pasta.

Food regulations of many territories, such as North America, require packaged food products to be labeled and claims of nutritional delivery to not be misleading. The recommended daily intake of fruits and/or vegetables varies somewhat based on daily caloric needs and age but is generally between 3 to 5 servings. USDA food composition tables for the U.S. and the Canadian Nutrient Files provide data on the nutrient composition of standard fruits and vegetables, juices and purees. The principle underlying the present invention is to provide at least the minimum level of key nutrients expected to be found in a food preparation based on the food composition tables. The process of the present invention includes analysis of minimum, average and maximum levels of key nutrients expected to be found in a food preparation based on the food composition tables, and allows discretion to vary servings assessments within these ranges. The key nutrient assessment is based on the fresh fruits and vegetables used to prepare the food preparation. For example, if 3 servings of fruits and vegetables are claimed, then the nutrients provided in the food preparation should be at a level consistent with 3 servings of vegetables.

Thus, a nutritional target comprising a number of servings of a particular food is determined as set by a food regulatory body. Nutrient benchmarks are developed, specific to each formulation/variety and new benchmarks are required for new variants. New products are then developed to meet the benchmarks based on nutritional density. For example, a dehydrated or chilled side dish or meal may seek to deliver one or two vegetable servings. A nutrient approach is particularly useful in those territories where there is no specific regulation as to what volume of dehydrated vegetables constitutes a serving.

The next step in the inventive process is determining key nutrients in the particular food, such as fruits and vegetables.

Determining the Key Nutrients

As a step in setting recommended eating patterns for fruits and vegetables the key dietary nutrients provided from fruit and vegetable intake are determined and classified as major contributors (about 20% or more of the daily value) or substantial contributors (greater than about 10% of the daily value), of which an example is shown in the Table below. Both the major and substantial contributors are treated as key nutrients for purposes of the inventive process depending on the contribution of the nutrient to the daily value found in a particular fruit or vegetable used in a product formulation. Both major and substantial nutrients are considered to be important if they are expected to provide 2% or more of the daily value for the nutrient in the product formulation. Note, depending on a particular mix of fruits and vegetables consumed in a particular region or by a particular population, or contribution from other foods that contribute to this list of nutrients (e.g., legumes), a substantial contributor may become a major contributor in any given case. The range of each of the key marker nutrients in the Table below was determined from the U.S. Dietary Guidelines Advisory Committee (DGAC) scientific Report issued in 2005. See http://www.health.gov/dietaryguidelines/dga2005/report/default.htm.

TABLE 1
Dietary Nutrient Contributions from Fruits and Vegetables

| Food Group | Major Contributor | Substantial Contributor |
|---|---|---|
| Fruit Group | Vitamin C | Thiamin, B6, Folate, |
| | | Magnesium, Copper, |
| | | Potassium and Fiber |
| Vegetable Group | Beta Carotene (Vit. A), | Vitamin E, Vitamin C, |
| | B6, Potassium, Copper, | Thiamin, Niacin, Folate, |
| | Fiber | Calcium, Phosphorus, |
| | | Magnesium, Iron and |
| | | Zinc |

A similar analysis may be performed for other food regulator body recommended foods categories.

Quantification of Fruit and Vegetables

The fruit and vegetable preparations for purposes of the present invention are prepared from between about 3 and about 5 portions of fresh fruits and vegetables depending on the particular variety. The actual volume of fruits and vegetables delivered in the packaged preparation may be less than what would be considered a serving under dietary regulations. However, if the key nutrients found in fresh fruits and vegetables are retained and delivered into the packaged products, then the amount of nutrients found in the preparation would be equivalent to the amounts found in between 3 and 5 servings of fruits and vegetables.

### Step 1: Reference the Nutrient Content of Fresh Fruits and Vegetables

A list of the fruits and vegetables used to prepare each variety of a food preparation is created. Nutrient contents of various fruit and vegetable forms are referenced in the DGAC Report at http://www.health.gov/dietaryguidelines/dga2005/report/HTML/D1_Tables.htm as exemplary for pumpkin, lemon, acerola, apple, apple concentrate, banana, carrots, sweet corn, kiwi, orange, passion fruit, pectin, pineapple, strawberry. A range of low, medium and high nutrient content is obtained.

Step 2: Determine which are the Key Nutrients for Each Variety Formula

The final product should deliver equal to or above the minimum expected value for each nutrient based on the target number of portions or servings of fruits and vegetables, for example, 1-3 servings. Therefore the minimum level of each target nutrient of the fruit and vegetables in a fruit and vegetable preparation formula (based on USDA National Nutrient Database at http://www.nal.USDA.gov/fnic/foodcomp/search/index.html or Canadian Nutrient Files) was selected for all calculations. Only nutrients which are expected to be at 2% or more of the daily value at the 1 or more serving's level for a formula is to be used for the substantiation. This is based on a view that any nutrient from Table 1 that is below the 2% daily value is not significant from a dietary point of view.

This step of determining key nutrients can also be done by analysis, especially for those fruits and/or vegetables for which there is no tabular data, such as purple carrots, sea buck thorn, and other more exotic fruits and vegetables. A standard analytical method accepted in the industry may be use, such as authorized methodologies by the AOAC found at http://www.aoac.org.

### Step 3. Analyze Products for Key Nutrients Identified in Step 2 of Each Variety

Select nutrients from a number of production runs are determined using analytical methods consistent with industry standard. The average values for each nutrient from representing a number of production lots has been used for the following examples.

In the following, several examples of application of the inventive system and method are described. The following is by way of example, not by way of limitation, of the principles of the invention to illustrate the best mode of carrying out the invention.

### EXAMPLE 1

This example demonstrates the determination of Servings of Fruits and Vegetables (F&V) in drinkable fruit and vegetable preparations packaged in a container having less than ½ cup volume, based on Nutrient Density.

Table 2 shows the amount of raw fruit and/or vegetable needed to prepare a container of the fruit and vegetable preparation, although the package has an overall volume of less than ½ cup, i.e. about 3.4 fluid ounces of purposes of this Example.

A Banana Pumpkin Kiwi preparation composition is set forth in the Table below.

TABLE 2
Banana Pumpkin Kiwi

| | F&V to make 1 container |
|---|---|
| Pumpkin | 1 slice |
| Bananas | 3 slices |
| Kiwi | 1 slice |
| Carrot | ½ |
| Orange | 1 |
| Balance (other ingredients | |
| not counted toward | |
| nutritional density) | |

The following steps were undertaken:

The nutrient content of fruits and vegetables used to prepare each variety of a food preparation was determined as per Step 1 above, i.e., nutrient content per food composition tables found in USDA National Nutrient Database.

The expected level of key nutrients in each variety of a food preparation was determined based on the formulation at a 3 serving's nutrient level, as per Step 2 above. The nutrient list was adjusted to include only those nutrients above 2% of the daily value. For example, for the Potassium, Vitamin C, and Vitamin B6 were delivered at over 10% of daily value; Vitamin A, Thiamin, Folate, and Magnesium were delivered at 5-10%.

The amount of each target nutrient at the 3 serving's level was compared to the amount in the fruit and vegetable preparation determined by standardized analytical testing For example, for the Banana Pumpkin Kiwi variety, 3 USDA servings would deliver about 69 mg Vitamin C, while actual product delivered about 84 mg Vitamin C; 3 USDA servings would deliver about 453 mg Potassium while the actual product delivered about 553 mg; 3 USDA servings would deliver about 7577 mg Vitamin A while the actual product delivered about 9244 mg; 3 USDA servings of Iron would deliver about 0.74 mg while the actual product delivered 0.90 mg. See the table below.

TABLE 3
Nutrients in Banana Pumpkin Kiwi Preparation

| | Vitamin | Potassium | Vitamin | Iron | Fiber | Ca |
|---|---|---|---|---|---|---|
| | C (mg) | (mg) | A (IU) | (mg) | (g) | (mg) |
| Nutrients in | 69 | 453 | 7577 | 0.74 | 4.3 | 54 |
| 3 USDA | | | | | | |
| Servings | | | | | | |
| Nutrients in | 84 | 553 | 9244 | 0.90 | 5.3 | 65 |
| Preparation | | | | | | |
| as measured | | | | | | |
| Analytically | | | | | | |

Equivalents to 3 Fruit and vegetable servings were calculated, as follows:
Formula water and other ingredients not deemed to directly contribute to nutrition density calculations, such as orange pulpy were removed from the formulas and the percentage of each of the remaining ingredient was normalized to a total of 100%.
For each target nutrient in Table 1 the amount provided from a USDA serving of each ingredient was multiplied by 3 to give the amount of that nutrient in 3 servings of the fruit or vegetable ingredient. The resulting number was then multiplied by the corrected percentage in the formula determined in step 1 above giving the expected contribution of that nutrient from 3 servings of the ingredient. This result was the development minimum for that nutrient and the reference point for comparison to the analytical data.
The total for each nutrient was then evaluated against the daily value and any nutrient falling below 2% of the daily value was removed from the substantiation case and not used as a target nutrient for nutrient density analysis or as a product development.

As per Step 3 above, product were analyzed analytically for key nutrients identified in Step 2, for each variety. An evaluation of how close the measured nutrient levels in the fruit and vegetable preparations are to the levels expected from 3 servings of fruits and vegetables. The results showed that a claim of 3 servings of fruits and vegetables can be substantiated based on nutrient density.

Note, that, preferably, this level for each nutrient should be found at the end of shelf life.

While the present invention has been described herein with some specificity, and with reference to certain preferred embodiments thereof, those of ordinary skill in the art will recognize numerous variations, modifications and substitutions of that which has been described which can be made, and which are within the scope and spirit of the invention. It is intended that all of these modifications and variations be within the scope of the present invention as described and claimed herein, and that the inventions be limited only by the scope of the claims which follow, and that such claims be interpreted as broadly as is reasonable. Throughout this application, various publications have been cited. The entireties of each of these publications are hereby incorporated by reference herein.

What is claimed is:

1. A method for determining number of fruit and vegetable equivalent servings of concentrated fruit and vegetable food preparation comprising:
a. providing a volume of said preparation that is up to about ½ cup;
b. measuring nutrient density of said volume;
c. comparing said nutrient density to that in food regulatory body recommended fruit and vegetable servings;
d. Claiming the appropriate equivalent fruit and/or vegetable servings.

2. The method of claim 1, wherein said fruit and vegetable food preparation is drinkable.

3. The method of claim 1, wherein the number of servings of fruit and/or vegetable is about 3 to about 5.

4. The method of claim 1, wherein the food regulatory body is USDA.

5. The method of claim 1, wherein one or more of said volumes is provided in a package.

6. A method of formulating a packaged food product comprising nutrient containing food ingredients to provide servings of a particular food group based on nutritional requirements of regulatory bodies, said method comprising:
a. ascertaining nutritional regulations for at least one nutrient;
b. measuring the content of said at least one nutrient in said food product;
c. adjusting the amount of one or more of said food ingredients so as to meet said nutritional regulations for said at least one nutrient;
d. claiming on the package the equivalent said food group servings.

7. The method of claim 6, wherein said food product is a fruit and vegetable preparation, a dehydrated side dish, or chilled side dish or meal.

8. The method of claim 6, wherein said food group is selected from the group consisting of fruit, vegetable, fat, oil, grain, carbohydrate, and meat.

9. The method of claim 6, wherein the number of servings of fruit and/or vegetable is about 3 to about 5.

10. The method of claim 6, wherein said claiming is based on an equivalent to a freshly picked food.

11. The method of claim 6, wherein said claiming is based on an equivalent to the maximum values for nutrients in USDA National Nutrient Database or those of other food regulatory bodies.

12. A method of formulating a food composition to a nutritional target by selecting food ingredients having a plurality of nutrients therein, comprising:
(a) Setting a nutritional target comprising number of servings of a food regulatory body recommended food;
(b) Determining the amount of each nutrient expected from said nutritional target from a food from food composition tables of a food regulatory body;
(c) Adjusting the nutrient list to include only those nutrients above 2% of the daily value based on said food composition tables, thereby determining a key nutrient list; and
(d) Determining an amount of each key nutrient needed to meet said nutritional target;
(e) Measuring the amount of said key nutrients in said food composition by standardized analytical testing;
(f) Adjusting the food composition amount of food ingredients to meet said nutrition target based on said analytical testing.

13. The method according to claim 12, wherein said target comprises one or more servings of said recommended food.

14. The method according to claim 12, wherein said target is one or more fruit and/or vegetable servings.

15. The method of claim 12, wherein said food composition tables are found in the USDA National Nutrient Database or that of other food regulatory bodies.

16. A method for determining the number of fruit and/or vegetable servings in a packaged dehydrated or frozen food composition containing fruit and/or vegetable pieces, said method comprising:
a. providing said composition having a volume of said fruit and/or vegetable pieces that is up to about ¼ cup;
b. measuring the nutrient density of said volume;
c. comparing said nutrient density to that in a food regulatory body recommended fruit and vegetable servings;
d. claiming on the package the appropriate equivalent of fruit and/or vegetable servings.

17. The method of claim 16, wherein the number of said fruit and/or vegetable servings is about 1 to about 5.

18. The method of claim 16, wherein the food regulatory body is USDA and/or Canada Food Guide or other food regulatory bodies of respective territories.
